(19) United States
(12) Patent Application Publication
(10) Pub. No.: US 2010/0261157 A1
(43) Pub. Date: Oct. 14, 2010

(54) Device and Method for Processing a Sample Contained in a Swab for Diagnostic Analysis

(75) Inventor: David A. Brock, Elkhart, IN (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Healthcare Diagnostics Inc., Tarrytown, NY (US)

(21) Appl. No.: 12/747,293

(22) PCT Filed: Dec. 9, 2008
(86) PCT No.: PCT/US08/86049

§ 371 Date: Jun. 10, 2010

Related U.S. Application Data
(60) Provisional application No. 61/012,868, filed on Dec. 11, 2007.

(51) Int. Cl. C12Q 1/70; C12M 1/00; C12Q 1/02; B23P 11/00

(52) U.S. Cl. 435/5; 435/283.1; 435/29; 29/428

(57) ABSTRACT

A device for processing a sample contained in a swab for diagnostic analysis, includes a chamber having a first chamber portion and a second chamber portion to receive the swab and a processing fluid, wherein at least one of the first and second chamber portions is flexible. A divider may be positioned in the chamber to facilitate transferring the sample to the second chamber portion, and a delivery channel is disposed in fluid communication with at least one of the first chamber portion and the second chamber portion to deliver a processed sample for diagnostic analysis.

### FIELD OF INVENTION

The present invention relates to devices and methods for diagnostic analysis. More particularly, the invention relates to a device, method, and a kit for processing a sample contained in a swab.

### BACKGROUND OF INVENTION

Diagnosis of biological samples has gained increased importance in medical treatment and practice. Rapid identification of disease-causing organisms in biological samples is very important. Several diagnostic tests exist for detecting analytes in biological samples, within a period of minutes, as opposed to the traditional period of days involved with bacterial cultures. For example, there are devices for testing Group A Streptococcus (Strep A) within minutes, which allow quick diagnosis and treatment.

In many instances, to carry out a diagnostic test such as Strep A, the operator inserts a swab containing a sample into a flexible test tube and then manually squeezes the tube to remove the sample from the swab. After the swab is removed from the test tube, the tube is capped with a dropper cap. The capped tube is inverted and the sample is then applied to the diagnostic test device. This approach thus involves the use of manual steps that are prone to user error, and the assembly of discrete components, which, if performed incorrectly, may result in spillage, contamination, and/or the possibility of the user being exposed to biohazardous materials.

Another approach is disclosed in U.S. Pat. No. 5,415,994, entitled “Lateral Flow Medical Diagnostic Assay Device with Sample Extraction means”; Imrich et al, issued May 16, 1995, which is fully incorporated herein by reference. The Imrich patent discloses a device, method, and kit for detection of analytes in samples. This device includes an open extraction chamber, into which a user places a sample-containing swab. The user then pours an extraction fluid over the swab. Extracted sample is removed from the extraction chamber through an exit port, and is then passed to a sample receiving zone and subsequently tested. Thus, the sample is initially extracted in an open container, and then removed from this primary container prior to application to a test device.

This approach too, however, has drawbacks. For example, sample extraction involves user-implemented steps that are subject to error, presenting opportunities for inaccuracies, contamination, and/or exposure to biohazardous materials. In this regard, incomplete coverage of the swab with extraction fluid may adversely affect sample extraction, while the open nature of the extraction container during the extraction steps presents opportunities for sample contamination by foreign objects and/or spillage to potentially expose the user to biohazardous materials. Moreover, there has recently been concern that a Clinical Laboratory Improvement Amendments (CLIA) waiver would not be available for a diagnostic test if the extracted sample were removed from the primary (extraction) container prior to delivery of the sample to the test device, ostensibly due to the aforementioned contamination and exposure concerns.

Thus, a need exists for a single device that allows both the extraction of the sample from the swab, and delivery to a diagnostic test device, in a single, closed container, while reducing the risk of user error, sample contamination, and/or exposure to biohazardous material.

### SUMMARY OF THE INVENTION

According to an embodiment of the invention, a device for processing a sample contained in a swab for diagnostic analysis comprises a chamber comprising a first chamber portion and a second chamber portion for receiving the swab and a processing fluid, wherein at least one of the first and second chamber portions is flexible; a divider is positioned in the chamber and is able to transfer the sample to the second chamber portion, and there is a delivery channel in fluid communication with at least one of the first and second chamber portions, to deliver a processed sample for diagnostic analysis.

Another embodiment includes a method of processing a sample contained in a swab for diagnostic analysis, the method including introducing the swab into a first chamber portion of a chamber, and passing the swab through a divider in the chamber to transfer the sample into a second chamber portion of the chamber, so that the second chamber portion contains a processing fluid. The method also includes displacing a processed sample through a delivery channel in fluid communication with at least one of the first and second chamber portions for diagnostic analysis.

Another embodiment includes a diagnostic kit for analyzing a sample, the diagnostic kit comprising a device as described above, at least one processing fluid, and a diagnostic test for analysis.

Still another embodiment includes a method of fabricating a device for processing a sample contained in a swab for diagnostic analysis, the method including providing a chamber having a first chamber portion and a second chamber portion for receiving the swab and a processing fluid, in which at least one of the first and second chamber portions is flexible. The method also includes placing a divider in the chamber to transfer the sample for processing, and providing a delivery channel in fluid communication with at least one of the first chamber portion and the second chamber portion to deliver a processed sample for diagnostic analysis.

### BRIEF DESCRIPTION OF THE FIGURES

FIG. 1 is an elevational, schematic view of a device for processing a sample contained in a swab in accordance with an embodiment of the invention.

FIG. 2 is a cross-sectional view, taken along 2-2 of FIG. 1.

FIG. 3A is a view similar to that of FIG. 1, without a swab.

FIG. 3B is a view similar to that of FIGS. 1, 3A, with the swab in a second chamber portion in accordance with an embodiment of the invention.

FIG. 3C is a view similar to those of FIGS. 1, 3B, with the swab removed and retained in a first chamber portion in accordance with an embodiment of the invention.

FIG. 3D is a view similar to those of FIGS. 1, 3B, 3C, with the second chamber portion squeezed in accordance with an embodiment of the invention.

FIG. 4 depicts a flowchart for a method of processing a sample contained in a swab.

FIG. 5 illustrates a diagnostic test in accordance with an embodiment of the invention.

### DETAILED DESCRIPTION

The present invention relates to a device, method, and kit for processing a sample contained in a swab for diagnostic analysis.

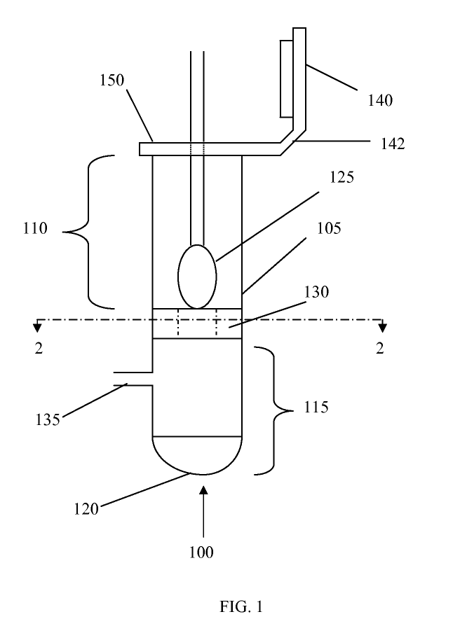

According to an aspect of the invention, as shown in FIG.1, a device 100 for processing a sample contained in a swab 125 for diagnostic analysis includes a chamber 105 having a first chamber portion 110 and a second chamber portion 115 for receiving the swab 125 and a processing solution (such as a conventional extraction or lysing fluid) 120, wherein at least one of the first chamber portion 110 and the second chamber portions 115 is flexible. It is noted that the processing solution 120 may be placed into the device by the user, or alternatively, the device 100 may be pre-loaded with solution 120, e.g., by the supplier or manufacturer. The device 100 includes a divider 130 positioned in the chamber 105 and is able to transfer the sample to the second chamber portion 115, and a delivery channel 135 which is in fluid communication with the second chamber portion 115, to deliver a processed sample for diagnostic analysis. Optionally, the delivery channel 135 may be in fluid communication with the first chamber portion 110. The first chamber portion 110 receives the swab 125 through an opening 150 disposed therein. The swab 125, which contains the sample, is then passed through the divider 130 to transfer the sample into the second chamber portion 115, which contains the processing fluid 120.

Generally, the sample will be biological material obtained or derived from a patient or non-patient sample. Biological samples derived from patients can include physiological material such as urine, serum, cerebrospinal fluid, nasal secretions, gastric secretions, sputum, pharyngeal exudates, urethral or vaginal secretions, and the like. Non-patient samples can include microbial contamination of immortalized cell lines, food stuffs, agricultural products, and the like. In some cases, samples may also include non-biological samples. The processing solution (e.g., extraction or lysing fluid) generally is a solution including reagents which will treat the sample to enhance the detection of an analyte or antigen in the sample. Suitable processing solutions may include acids, detergents or chelators. In particular examples, for the immunological detection of Group A streptococcus by devices of the present invention, a swab containing a sample of pharyngeal exudate may be pretreated with an acidic processing solution, such as nitrous acid, to expose Group A streptococcus-specific antigens. Alternatively, to detect Legionella pneumophila by immunochemical means, a swab containing a sputum sample may be pretreated with a processing solution containing a Triton X-100 detergent and EDTA in phosphate-buffered saline.

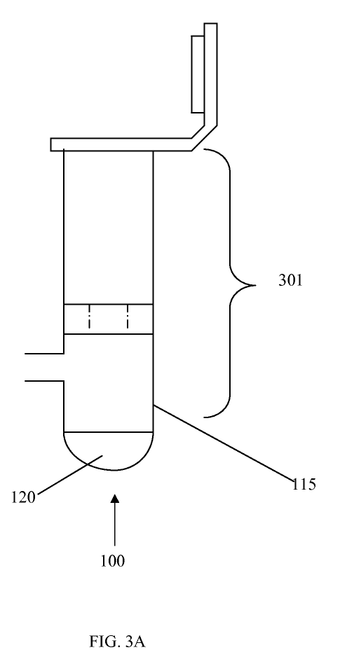

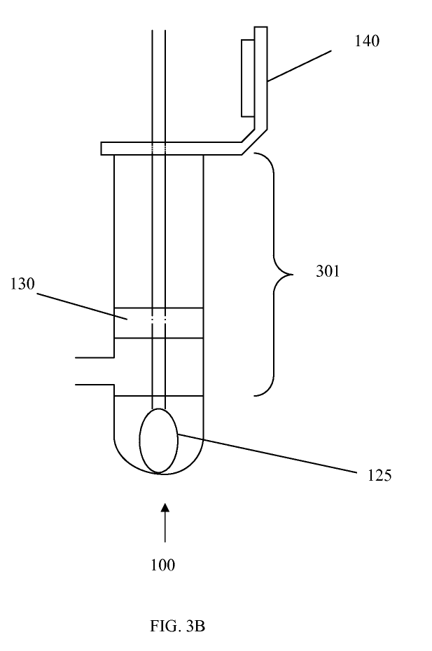

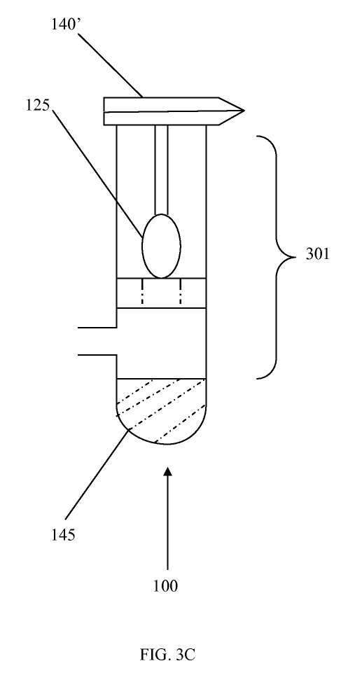

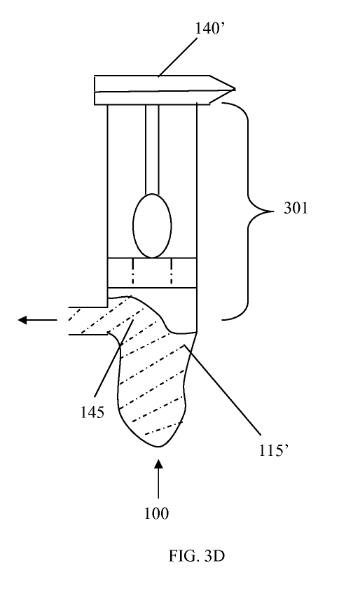

Optionally, various embodiments may include a cap 140 coupled (e.g., with an integral hinge 142) to the first chamber portion 110 of the device and able to seal the opening 150 of the device in a fluid-tight (e.g., liquid- and air-tight) manner. According to another aspect of the invention, the first chamber portion 110 is relatively more rigid than the second chamber portion 115. According to another embodiment, the second chamber portion 115 is made of flexible plastic, such that the user can squeeze the second chamber portion 115 (e.g., as shown in FIG. 3D) to deliver the processed sample through the delivery channel 135.

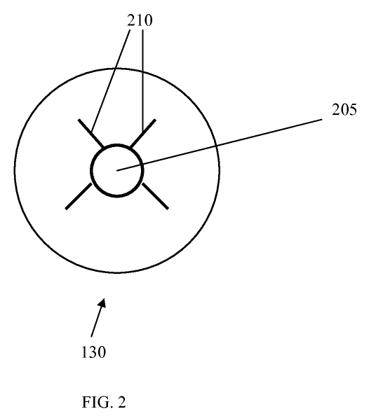

As shown in FIG. 2, the divider 130 comprises an aperture 205 through which the swab 125 may be passed into the second chamber portion 115. Furthermore, according to another aspect, the divider 130 can be designed to function as a squeegee, wherein the divider squeezes the sample from the swab 125 into the second chamber portion 115, as the user passes the swab 125 through the divider upon insertion and/or upon removal of the swab from the second chamber portion 115. In this regard, the aperture 205 may be sized to provide an interference with the swab 125 as it passes therethrough. The divider may be fabricated from a resilient material such as a suitable polymeric (plastic) material, optionally including a plurality of slits 210 therein. The resilient material, alone or in combination with the slits 210, enable the aperture 205 to expand to permit the swab to pass therethrough, while effectively squeezing the swab (to remove sample therefrom) as the swab is passed through aperture 205, e.g., into and out of the second chamber portion 115. It should be noted that the sample may include nominally any analyte for which testing may be performed, including various antigens and analytes such proteins, minerals, pH, etc. In particular embodiments, the analyte includes an antigen selected from the group consisting of Group A Streptococcus antigen, Group B Streptococcus antigen, Group C Streptococcus antigen, Pseudomonas aeruginosa antigen, Chlamydia trachomatis antigen, Neisseria gonorrhea antigen, Legionella pneumophila antigen, and herpes simplex virus antigen.

Use of the divider to remove the sample from the swab eliminates the need for a user to manually squeeze the sample out of the swab, to effectively remove user variability and the associated potential for user error from this sample processing operation. Also, after processing, the sample is ready for delivery, via the delivery channel, to a testing device for diagnostic analysis. Thus, the user is not required to move the sample to an intermediate chamber prior to testing.

Embodiments of the present invention thus eliminate steps prone to user error, and provide a single, closeable container for both sample processing/extraction and delivery to a test device, while optionally capturing the swab therein, for substantially reduced potential for inaccuracies, contamination, and/or exposure to biohazardous materials. The relatively simple, unitary nature of these embodiments may also facilitate low cost manufacture, use, and disposal of the device.

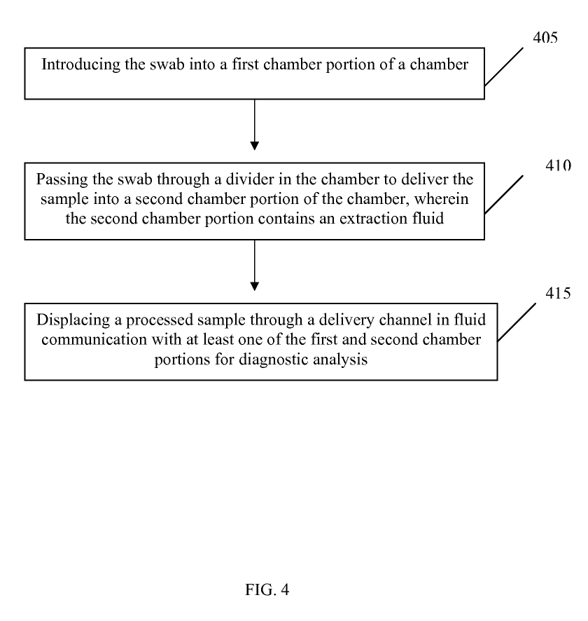

According to another embodiment, as shown in FIG. 4 a method of processing a sample contained in a swab for diagnostic analysis includes: introducing the swab containing the sample into a first chamber portion of a chamber as at 405, passing the swab through a divider in the chamber to transfer the sample into a second chamber portion of the chamber containing a processing fluid as at 410 and displacing a processed sample through a delivery channel in fluid communication with at least one of the first and second chamber portions for diagnostic analysis as at 415. The divider 130, as described earlier, includes an aperture 205 through which the swab 125 may be passed into the second chamber portion 115 of the device 100. The divider 130 may also be designed to function as a squeegee so as to squeeze the sample out of the swab 125.

In a particular embodiment, the displacing 415 further includes squeezing the second chamber portion 115 to displace the processed sample. The second chamber portion 115 can be made of flexible plastic to allow a user to squeeze the second chamber portion 115 to displace the processed sample. The method may further include withdrawing the swab from the second chamber portion 115 after processing the sample. Once the sample is extracted with the processing fluid, the swab can be removed or the swab can be broken off and retained within the first chamber portion 110. In this regard, the swab may be captured in the first chamber portion 110, such as by using the cap 140 to close the first chamber portion 110, to manage biohazardous waste and reduce the risk of contamination.

According to embodiments of the present invention, a decrease in the internal volume of the device enables the delivery of the sample for diagnostic analysis, as illustrated in FIGS. 3A, 3B, 3C, and 3D. Initially, as shown in FIG. 3A, when the processing solution 120 is added to the second chamber portion 115 of the device 100, the original air space or the internal volume 301 is as shown. A swab 125 containing the sample is introduced into the device through a divider 130 and brought into contact with the processing solution 120, as shown in FIG. 3B and the internal air volume 301 in the chamber reduces slightly. The swab may then be withdrawn from the second chamber portion 115 and a cap 140 coupled to the first chamber portion 110 seals the device, e.g., while capturing the sample-containing swab 125 therein, as shown in FIG. 3C. In this regard, after withdrawing the swab from the second chamber portion 115 into the first chamber portion 110, the user may conveniently break the handle of swab 125, e.g., along a score line or perforation provided therein, either prior to, or in conjunction with closing cap 140, to capture the swab 125 therein as shown. The second chamber portion now contains a processed sample 145. The second chamber portion, which may be relatively flexible, is squeezed by a user, and the internal air volume 301 is reduced, as shown at 115′ in FIG. 3D. This reduced internal volume 301, by virtue of the closed cap 140′, serves to increase the internal pressure sufficiently to expel the processed sample 145 through the delivery channel 135, e.g., for being deposited onto a diagnostic test strip, cassette, or other testing apparatus.

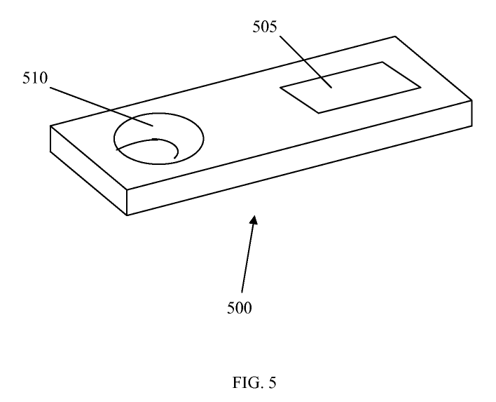

According to an embodiment, a diagnostic kit for analyzing a sample comprises a device 100, as described earlier and at least one processing fluid. The processing fluid includes reagents which will treat the sample to enhance the detection of an analyte or antigen in the sample. In particular embodiments, the sample may contain, for example, an antigen selected from the group consisting of Group A Streptococcus antigen, Group B Streptococcus antigen, Group C Streptococcus antigen, Pseudomonas aeruginosa antigen, Chlamydia trachomatis antigen, Neisseria gonorrhea antigen, Legionella pneumophila antigen, and herpes simplex virus antigen. The kit may further include a diagnostic test such as a diagnostic cassette 500 shown in FIG. 5. The diagnostic cassette includes an entry point 510 for introducing the sample, and a window 505 for viewing the results of the test.

In another embodiment, the invention includes a method of fabricating a device for processing a sample contained in a swab for diagnostic analysis, including providing a chamber comprising a first chamber portion and a second chamber portion for receiving the swab and a processing fluid, in which at least one of the chamber portions is flexible. The method also includes placing a divider in the chamber to transfer the sample for processing, and providing a delivery channel in fluid communication with at least one of the first chamber portion, and the second chamber portion to deliver a processed sample for diagnostic analysis. According to another embodiment, the method further includes coupling a cap to the first chamber portion to seal the device.

It should be understood that any of the features described with respect to one of the embodiments described herein may be similarly applied to any of the other embodiments described herein without departing from the scope of the present invention.

The foregoing description is intended primarily for purposes of illustration. Although the invention has been shown and described with respect to an exemplary embodiment thereof, it should be understood by those skilled in the art that the foregoing and various other changes, omissions, and additions in the form and detail thereof may be made therein without departing from the spirit and scope of the invention.

What is claimed is:

1. A unitary device for processing a sample contained in a swab for diagnostic analysis, and delivering the processed sample, the device comprising:
a chamber having a first chamber portion and a second chamber portion to receive the swab, wherein at least one of the first and second chamber portions is flexible;
and,
a delivery channel in fluid communication with at least one of the first chamber portion and the second chamber portion, to deliver a processed sample for diagnostic analysis.

2. The device of claim 1, further comprising a divider positioned in the chamber and configured to transfer the sample to the second chamber portion.

3. The device of claim 1, wherein the chamber is configured to receive a processing fluid therein.

4. The device of claim 3, comprising the processing fluid disposed therein.

5. The device of claim 4, wherein the processing fluid comprises an acid, a detergent or a chelator.

6. The device of claim 1, further comprising a cap coupled to the first chamber portion, wherein the cap is able to selectively seal the first chamber portion.

7. The device of claim 1, wherein the divider comprises an aperture to transfer the swab into the second chamber portion.

8. The device of claim 1, wherein the aperture is configured to remove the sample from the swab.

9. The device of claim 1, wherein the first chamber portion is relatively more rigid than the second chamber portion.

10. The device of claim 1, wherein the sample comprises an antigen selected from the group consisting of Group A Streptococcus antigen, Group B Streptococcus antigen, Group C Streptococcus antigen, Pseudomonas aeruginosa antigen, Chlamydia trachomatis antigen, Neisseria gonorrhea antigen, Legionella pneumophila antigen, and herpes simplex virus antigen.

11. A method of processing a sample contained in a swab for diagnostic analysis, the method comprising:
introducing the swab into a first chamber portion of a chamber through an opening;
transferring the sample into a second chamber portion of the chamber; and
displacing a processed sample through a delivery channel in fluid communication with at least one of the first and second chamber portions for diagnostic analysis.

12. The method of claim 11, wherein said transferring comprises passing the swab through a divider in the chamber.

13. The method of claim 11, comprising disposing a processing fluid in the second chamber portion.

14. The method of claim 13, wherein the processing fluid comprises an acid, a detergent or a chelator.

15. The method of claim 13, further comprising:
after said introducing, sealing the opening with a cap coupled to the chamber.

16. The method of claim 13, further comprising:
withdrawing the swab from the chamber after said introducing.

17. The method of claim 13, further comprising:
retaining the swab in the chamber after said introducing.

18. The method of claim 13, wherein said displacing further comprises:
squeezing the second chamber portion to displace the processed sample.

19. The method of claim 13, wherein the sample contains an antigen selected from the group consisting of Group A Streptococcus antigen, Group B Streptococcus antigen, Group C Streptococcus antigen, Pseudomonas aeruginosa antigen, Chlamydia trachomatis antigen, Neisseria gonorrhea antigen, Legionella pneumophila antigen, and herpes simplex virus antigen.

20. A diagnostic kit for analyzing a sample, the kit comprising:
the device of claim 1; and
at least one processing fluid.

21. The diagnostic kit of claim 20, wherein the sample contains an antigen selected from the group consisting of Group A Streptococcus antigen, Group B Streptococcus antigen, Group C Streptococcus antigen, Pseudomonas aeruginosa antigen, Chlamydia trachomatis antigen, Neisseria gonorrhea antigen, Legionella pneumophila antigen, and herpes simplex virus antigen.

22. The diagnostic kit of claim 20 further comprising a diagnostic test for analysis.

23. A method of fabricating a device for processing a sample contained in a swab for diagnostic analysis, the method comprising:
providing a chamber comprising a first chamber portion and a second chamber portion, having an opening for receiving the swab and a processing fluid, wherein at least one of the first and second chamber portions is flexible; and,
providing a delivery channel in fluid communication with at least one of the first chamber portion and the second chamber portion to deliver a processed sample for diagnostic analysis.

24. The method of claim 23, comprising disposing a divider in the chamber to transfer the sample for processing.

25. The method of claim 16, further comprising coupling a cap to the device to seal the opening.
